(19) United States
(12) Patent Application Publication
(10) Pub. No.: US 2011/0144733 A1
(43) Pub. Date: Jun. 16, 2011

(54) Electrode for a Cochlear Implant

(75) Inventors: Fysh Dadd, New South Wales (AU); Claudiu Treaba, Englewood, CO (US)

(73) Assignee: Cochlear Limited, New South Wales (AU)

(21) Appl. No.: 13/023,287

(22) Filed: Feb. 8, 2011

Related U.S. Application Data
(63) Continuation of application No. PCT/AU2009/000871, filed on Jul. 7, 2009.

(30) Foreign Application Priority Data
Aug. 8, 2008 (AU) 2008904057

(51) Int. Cl. A61N 1/05; H01R 43/00

(52) U.S. Cl. 607/137; 29/592.1

(57) ABSTRACT

An electrode for a cochlear implant for implantation in a cochlea of a patient, the electrode having a first side that is curved in a lateral dimension so as to substantially conform to a side wall of a scala upon implantation of the electrode into a cochlea and a second side that is shaped so as to preserve a natural acoustic pathway within the scala upon implantation of the electrode into the cochlea. In one example, opposing ends of the electrode taper off in cross section. In another example, the fluid contact side is concave. In a further example, the electrode is one of crescent-shaped, oval shaped, and semi-circular in cross section. By using such an electrode, a more natural fluid-flow in the cochlea can be maintained after implantation relative to an original fluid-flow prior to implantation.

### CROSS REFERENCE TO RELATED APPLICATIONS

The present application is a continuation of PCT/AU2009/000871, filed on Jul. 7, 2009, and claims priority to Australian Patent Application AU 2008904057, filed in the Australian Patent Office on Aug. 8, 2008, the entire contents of both of which are incorporated herein by reference.

### TECHNICAL FIELD

This disclosure relates to electrodes for medical implants.

### BACKGROUND

It is not uncommon for a patient to experience partial hearing loss (for example due to damage to only a portion of the hair cells), and to retain some residual hearing. In such cases, if surgery to treat the partial hearing loss is performed, there is an accompanying risk that the residual hearing is negatively affected as a result of the surgery.

For example, physical damage may be caused to healthy tissue during the implantation process, or the mere presence of a foreign object in the cochlea may affect the dynamics of the hearing process. In the case of a cochlear implant in which an electrode is inserted into the cochlea of a patient, a short electrode may be implanted to replace the function of the damaged hair cells, but leave the unaffected hair cells (usually an apical portion). The mere presence of the short electrode in the scala tympani can, however, affect the distribution of energy generated by external sounds to the healthy hair cells.

Existing methods and implants attempt to address this problem by reducing the lateral cross-sectional area of the electrode to minimize disruption. Even a smaller electrode still interferes with a natural function of the cochlea.

### SUMMARY

According to one aspect of the present disclosure, there is provided an electrode for a cochlear implant for implantation in a cochlea of a patient, the electrode comprising an electrode contact side for supporting at least one electrode contact and a fluid contact side for exposure to fluid in the cochlea, wherein, the electrode contact side is curved in a lateral dimension so as to substantially conform to a wall of the cochlea upon implantation, and wherein the electrode is non-circular in cross section so as to maximize a naturally occurring cross-sectional fluid-flow area in the cochlea, minimizing disruption.

In one form, the fluid contact side is concave in cross section. In one form, the cross section is substantially oval. In one form, the cross section is substantially semi-circular. According to another aspect of the present disclosure, there is provided an electrode for a cochlear implant for implantation in a cochlea of a patient, wherein the electrode is shaped such that for a given cross-sectional area, the electrode, when implanted into the cochlea, provides for a more natural fluid-flow cross-sectional area in the cochlea than if the electrode were substantially circular in cross section.

### BRIEF DESCRIPTION OF THE DRAWINGS

The various aspects of the present disclosure are described in detail below, with reference to the following drawings in which:

FIG. 1—shows a graph of the velocity of fluid within a channel as a function of its distance from a wall of the channel;

FIG. 2A—shows the effective cross section of the scala tympani in a cochlea using a conventional electrode;

FIG. 2B—shows the corresponding effective cross section of the scala tympani in the cochlea using an electrode according to one aspect of the present disclosure;

FIG. 3—shows an electrode according to an aspect of the present disclosure located (perimodiolar) in the scala tympani of a cochlea;

FIG. 4—shows the arrangement of FIG. 3, showing the effective cross-sectional area for fluid flow;

FIG. 5—shows a “symmetrical” embodiment of the electrode of FIGS. 3 and 4;

FIG. 6—shows an embodiment of the electrode of FIGS. 3 and 4 with “wings”;

FIG. 7—shows an electrode having an elliptical configuration;

FIG. 8—shows an electrode having a modified elliptical configuration;

FIG. 9—shows an electrode having another modified elliptical configuration;

FIG. 10—shows a conventional circular non-perimodiolar electrode;

FIG. 11—shows a non-perimodiolar electrode according to one aspect of the present disclosure;

FIG. 12—shows a non-perimodiolar electrode design according to one aspect of the present disclosure;

FIG. 13—shows an alternative non-perimodiolar electrode design to that of FIG. 12;

FIG. 14—shows an elliptical non-perimodiolar electrode design;

FIG. 15—shows an alternative non-perimodiolar electrode design;

FIG. 16—shows yet a further alternative non-perimodiolar electrode design;

FIGS. 17A-17D—show an electrode as it traverses across the scala tympani;

FIGS. 18A-18F—show the change in effective cross-sectional area with change in the electrode cross sectional for a “winged” electrode shape;

FIGS. 19A-19F—show the change in effective cross-sectional area with change in the electrode cross sectional for a “symmetrical” electrode shape; and

FIG. 20—shows a perspective view of an electrode according to one aspect of the present disclosure, having a cross section as shown in FIG. 4.

### DETAILED DESCRIPTION

Throughout the following description, the phrase “cross section” will be used. It will be understood that “cross section” refers to the cross section taken substantially perpendicularly to the length of the electrode or the length of the scala tympani or other channel of the cochlea or other vessel such as a blood vessel, vein, or artery.

The term “effective cross-sectional area” as used in this specification is the area that remains in the cochlea, with the electrode in situ, that is available for free and natural movement of cochlear fluid. This may be represented in the figures by a circle, oval, or maximum-sized regular closed curve in the cross-sectional area that remains in the cochlea with the electrode in situ. The shape and cross-sectional area of the effective cross-sectional area (e.g., after implantation) of a particular portion of the cochlea, in comparison to the shape and cross-sectional area of that same portion before implantation, at least partially determines how much of the anatomical acoustic pathway is retained after implantation (and e.g., how “naturally” the fluid flows in the cochlea after implantation). While circles and ovals are used throughout the figures for ease of comparison purposes in relation to illustrating example effective cross-sectional areas, it should be understood that an effective cross-sectional area, in practice, may have a more irregular shape, and will be impacted by the shape of the cochlea and/or the electrode, among other things.

The word “traverse” in this specification is used to describe the region between the cochleostomy (or round window) and where the electrode becomes perimodiolar in the basal turn.

The design of the present disclosure, allows for an electrode that preserves residual hearing via a fundamentally different approach from that of the prior art. According to one aspect of the present disclosure, instead of focusing on reducing the negative impact of the electrode in the cochlear by simply decreasing the cross-sectional area of the electrode, the electrode of the present disclosure aims to instead focus on the shape and size of the effective cross-sectional fluid-flow area of the cochlea (e.g., relative to pre-implantation cross-sectional fluid-flow shape and size).

When a fluid flows through a pipe there are losses along the wall. The basic premise of pipe design is to maximize the volume and minimize the perimeter. There is effective friction on the perimeter of a pipe called a boundary layer. In the boundary layer the fluid flows at a lower velocity.

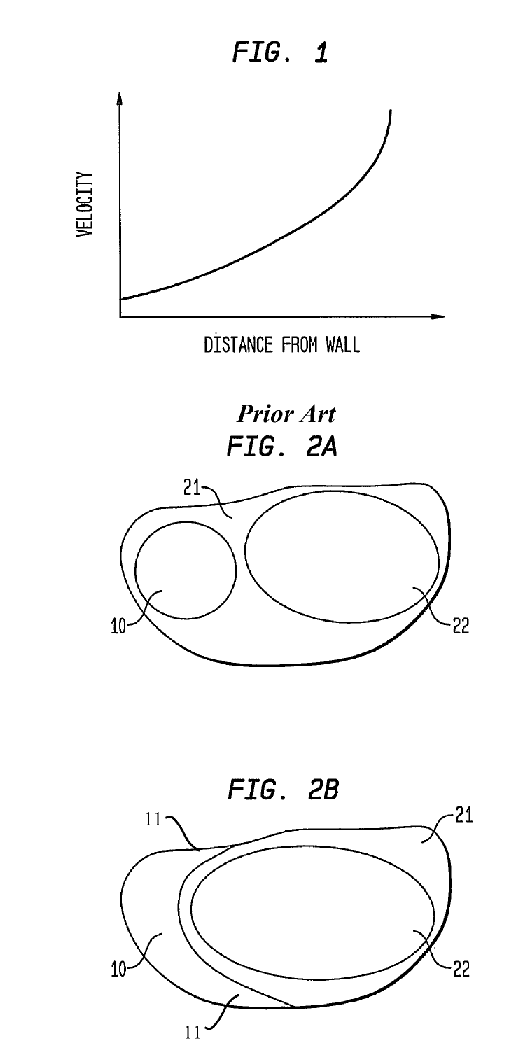

FIG. 1 shows a graph demonstrating the relationship between the velocity of fluid flowing through a pipe and the distance of the fluid from the wall of the pipe. As can be seen, the closer the fluid is to the wall of the pipe, the lower its velocity.

Movement within the fluid (perilymph) in the cochlea travels in the scala vestibuli to the helicotrema and down the scala tympani. This movement within the fluid moves the basilar membrane, Reissner's membrane, and tectorial. The tectorial membrane moves and touches the hair cells, resulting in cells firing and thus “hearing.”

The disclosed design allows fluid to move more naturally in the cochlea with the aim to keep residual hearing and provide for a more natural fluid-flow in the scala tympani after implantation relative to the fluid-flow prior to implantation.

A common complaint from recipients is that the sound they receive after an implant is tinnier (i.e., the sounds are of a higher frequency than they are used to). This is because, after an insertion of an electrode, the perceived hearing frequencies shift. This shift is caused by the impact of having an electrode occupying space in the cochlea that was previously empty. The electrode's presence causes a reduction in the effective cross-sectional area of the cochlea and, due to the circular shape of prior electrodes, a change in shape of the effective cross-sectional area. Natural sound (residual hearing) is still inputted to the patient having the same energy. The same energy with a smaller cross section and/or different shape results in a different wave frequency, resulting in stimulation at a different position(s) along the cochlea. Thus, according to this aspect of the disclosure, focus is placed on providing a more natural fluid-flow in the channel, rather than simply on reducing the cross-sectional area of the electrode. This is a significant departure from the prior art methods and designs of simply attempting to reduce the cross-sectional area of the electrode, without consideration of changes in pre- and post-implantation fluid flow characteristics in the channel.

Thus, in this aspect, the electrode design may maintain a same or similar physical cross-sectional area and thus amount of material (e.g., wire diameter and silicone volume required for shape retention) as before, but optimizes the size and shape of the effective cross-sectional area in the cochlea for fluid flow.

Referring to FIG. 2A, there is shown a cross section of a conventional electrode 10 in the scala tympani 21 of a cochlea (not shown in this view). FIG. 2B shows a cross section of an electrode according to one aspect of the present disclosure, in the scala tympani 21 of the cochlea. The circular electrode 10 of FIG. 2A has a same cross-sectional area as the improved electrode 10 of FIG. 2B, but the effective cross-sectional area 22 for the improved electrode design is 25% larger than the effective cross-sectional area using the design of FIG. 2A, and the shape of the remaining effective cross-sectional area is closer to the original shape of the cross-sectional area prior to implantation, allowing for a more natural fluid-flow within the cochlea.

Even as manufacturing methods improve and the volume of components required decreases, this improved design still allows for an electrode that provides the same functionality but that further increases the effective area in the cochlea for fluid flow and provides for more natural fluid-flow characteristics relative to fluid-flow characteristics of the cochlea prior to implantation.

This design may be used in any existing electrode configuration, including a Hybrid device, that is, one that has the electrode for cochlea stimulation and another means to facilitate residual hearing.

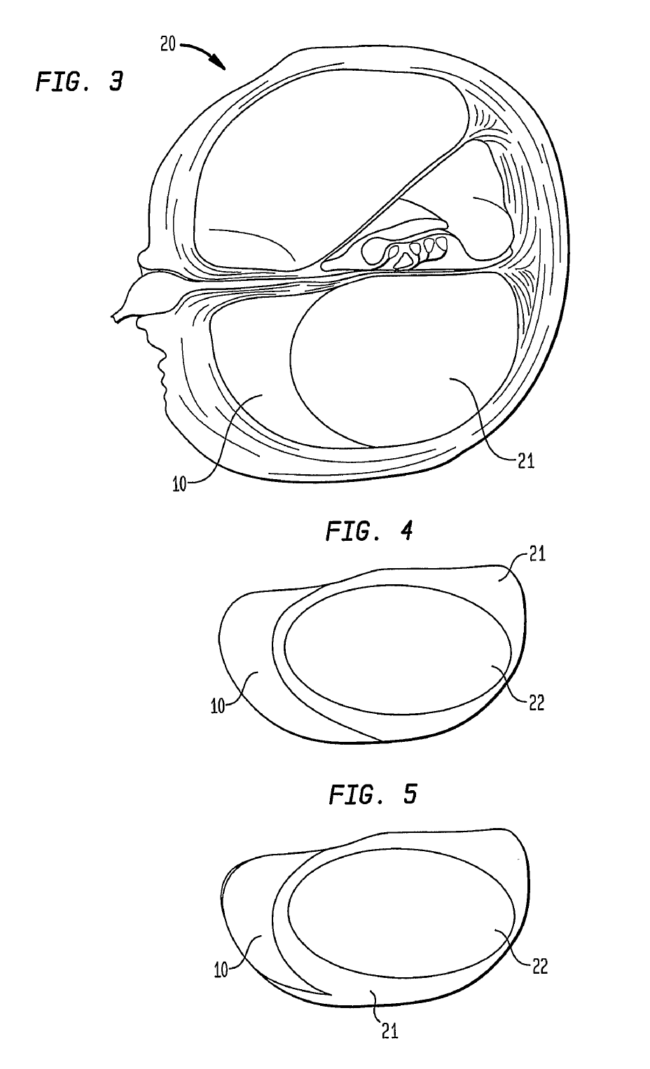

In one aspect of this design, for a perimodiolar electrode, the electrode is not only in gentle contact with the inner wall, but also the upper and lower walls. FIG. 3 shows a cross section of a cochlea 20 with an electrode 10 located in the scala tympani 21. The electrode 10 has a concave surface on its lateral wall. Additionally, the electrode 10 tapers at the tails (e.g., “wings” 11) to keep as close as possible to the shape of the cochlea 20 (e.g., in comparison to the shape of the cochlea prior to implantation).

FIG. 4 is a representation of the arrangement of FIG. 3 showing the scala tympani 21, the electrode 10, and the effective cross-sectional area 22. As can be seen, the effective cross-sectional area 22 is greater than it would have been had electrode 10 been of circular cross section and of the same cross-sectional area (e.g., compared to FIG. 2A). Furthermore, the effective cross-sectional area in the scala tympani 21 maintains a shape more similar to the scala tympani 21 prior to implantation.

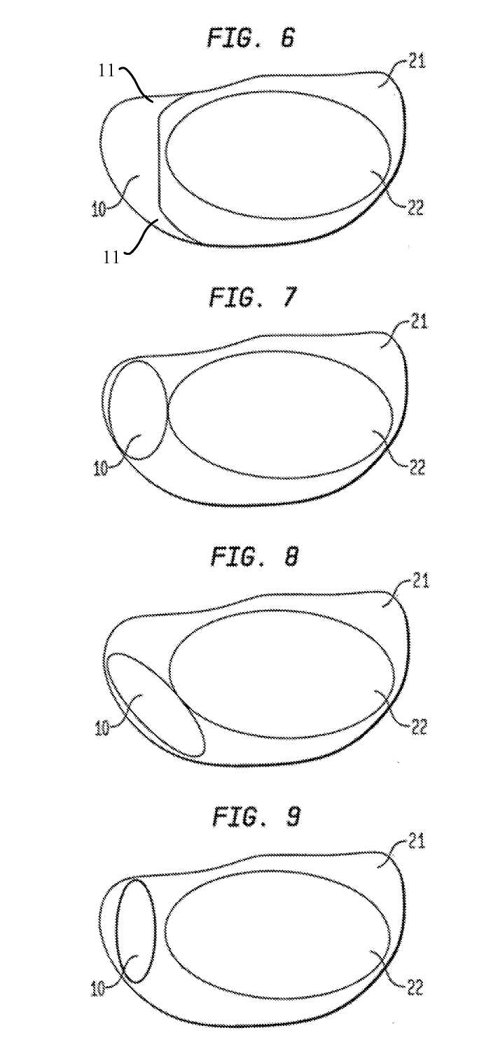

A number of different variations according to this aspect of the present disclosure are described with reference to FIGS. 5 to 9. It will be understood that they are described as separate embodiments only to provide clarity of each embodiment. However, two or more of the described embodiments may be combined. Furthermore, the various aspects of the present disclosure may be applied to full electrodes as well as to short electrodes.

FIG. 5 shows an embodiment of a “symmetrical” electrode 10 (e.g., symmetrical across a bisecting line). This design has the advantage of being relatively simple to manufacture and is able to be used in both the left and right cochlea. Again, the effective cross-sectional area 22 is shown in the scala tympani 21.

The electrode in FIG. 6 has upper and lower “wings” 11 extending from the body of electrode 10. These wings 11 may be formed of a soft material such as soft silicone LSR 30 and may be conformable to the walls of the scala tympani 21. This design may also be used in both the left and right cochlea. The wings 11 may be made such that a height/distance between wings 11 is slightly larger than the average cochlea so as to facilitate good contact with the walls. After insertion, the wings 11 would rest gently on the upper and lower wall of the scala tympani 21.

FIGS. 7-9 show embodiments in which electrode 10 has a substantially elliptical cross section. Again, it can be seen that the effective cross-sectional area of the scala tympani is greater than it would have been had the electrode 10 been of circular cross section with the same cross-sectional area, and the effective cross-sectional area has a shape and size (e.g., after implantation) that more closely mimics the shape and size of the scala tympani prior to implantation.

Table 1 below shows a comparison of the change in effective cross-sectional area using a “winged” substantially crescent-shaped configuration as compared to a circular configuration of the same cross-sectional area.

TABLE 1

| | | | | | Calc |
|---|---|---|---|---|---|
| | | | | | % increase |
| Figure on LHS | | | Figure on RHS | | in effective |
| (18A, 18C, and 18E) | | | (18B, 18D, and 18F) | | cross- |
| | | Remaining | | Remaining | sectional |
| | | effective | | effective | area (where |
| | | cross- | | cross- | electrode |
| Electrode | Electrode | sectional | Electrode | sectional | area is held |
| Diameter | Area | area | Area | area | constant) |
| 0.56 | 0.246 | 0.626 | 0.244 | 0.7824 | 25% |
| 0.685 | 0.369 | 0.526 | 0.369 | 0.719 | 36% |
| 0.87 | 0.595 | 0.322 | 0.597 | 0.582 | 81% |

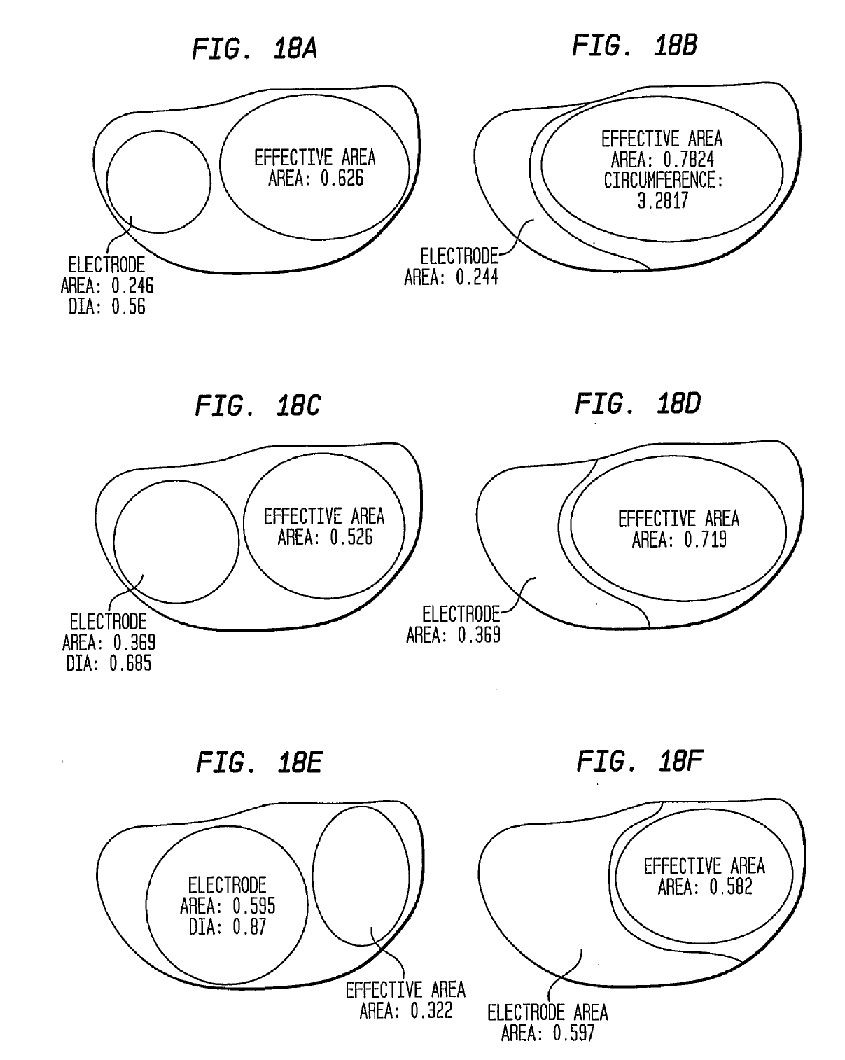

FIG. 18A shows a circular electrode of cross-sectional area of approximately 0.246 mm², leaving an effective cross-sectional area of approximately 0.626 mm². FIG. 18B shows the situation in which the circular electrode is replaced with a “winged” electrode (similar to FIG. 2B) according to an aspect of the present disclosure, of substantially the same cross-sectional area (0.244 mm²). As can be seen in FIG. 18B and Table 1 above, the effective cross-sectional area for fluid flow for the electrode of FIG. 18B is 0.7824 mm², an increase of 25% compared to the electrode of FIG. 18A.

FIGS. 18C and 18D show a similar situation in which the circular electrode has a cross sectional area of about 0.369 mm² and the “winged” electrode also has a cross-sectional area of 0.369 mm². In this situation, the “winged” electrode of FIG. 18D leaves an effective cross-sectional area of about 0.719 mm², a 36% increase compared to the electrode of FIG. 18C.

FIGS. 18E and 18F show a situation in which the cross-sectional areas of the electrodes are about 0.59 mm² (0.595 in the case of the circular electrode of FIG. 18E and 0.957 in the case of the “winged” electrode of FIG. 18F). In this situation, the “winged” electrode of FIG. 18F provides an effective cross-sectional area 81% larger than when the circular electrode of FIG. 18E is used.

In each of the above FIGS. 18B, 18D, and 18F, it is equally clear that, for a given electrode size, the shape of the effective cross-sectional area for fluid flow in the cochlea after implantation more closely mimics the shape of the effective cross-sectional area for fluid flow in the cochlea prior to implantation, leading to more natural fluid flow characteristics in the cochlea relative to the examples set forth in FIG. 18A, 18C, and 18E.

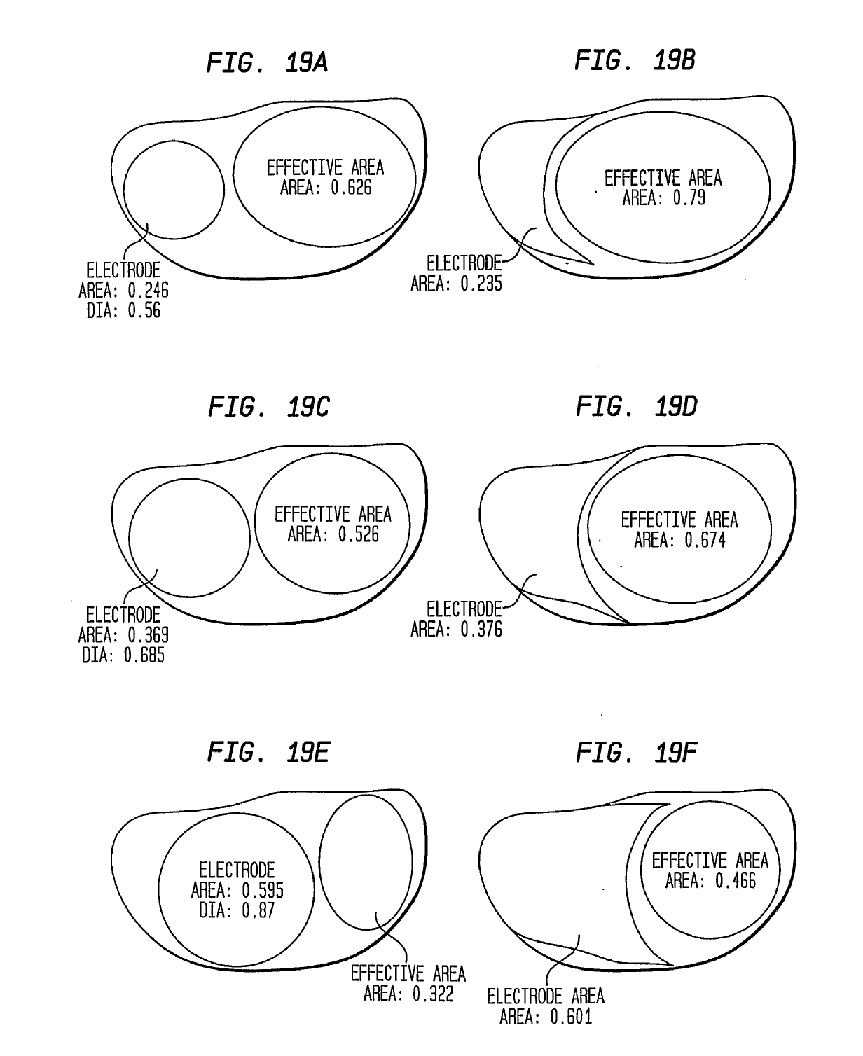

Table 2 and FIGS. 19A to 19F show a situation in which a “symmetrical” electrode according to an aspect of the present disclosure is used instead of a circular electrode.

TABLE 2

| | | | | | Calc |
|---|---|---|---|---|---|
| | | | | | % increase |
| Figure on LHS | | | Figure on RHS | | in effective |
| (19A, 19C, and 19E) | | | (19B, 19D, and 19F) | | cross- |
| | | Remaining | | Remaining | sectional |
| | | effective | | effective | area (where |
| | | cross- | | cross- | electrode |
| Electrode | Electrode | sectional | Electrode | sectional | area is held |
| Diameter | Area | area | Area | area | constant) |
| 0.56 | 0.246 | 0.626 | 0.235 | 0.79 | 26% |
| 0.685 | 0.369 | 0.526 | 0.376 | 0.674 | 28% |
| 0.87 | 0.595 | 0.322 | 0.601 | 0.466 | 45% |

FIG. 19A shows a circular electrode of cross-sectional area of about 0.246 mm², leaving an effective cross-sectional area of about 0.626 mm². FIG. 19B shows the situation in which the circular electrode is replaced with a “symmetrical” substantially crescent-shaped electrode according to an aspect of the present disclosure, having substantially the same cross-sectional area (0.235 mm²) as the circular electrode of FIG. 19A. As can be seen in FIG. 19B and Table 2 above, the effective cross-sectional area for fluid flow of the symmetrical electrode of FIG. 19B is 0.79 mm², an increase of 26% over the circular electrode of FIG. 19A.

These calculations may be made using any suitable techniques for calculating areas of geometric shapes including using well known formulae or commercially-available software CAD or other packages.

FIGS. 19C and 19D show a similar situation in which the circular electrode of FIG. 19C having a cross-sectional area of about 0.369 mm² is replaced with a “symmetrical” electrode of FIG. 19D having a substantially similar cross-sectional area of 0.376 mm². In this situation, the “symmetrical” electrode of FIG. 19D provides an effective cross-sectional area of about 0.674 mm², a 28% increase over the circular electrode of FIG. 19C.

FIGS. 19E and 19F show a situation in which the cross-sectional areas of the electrodes are about 0.59 mm² (0.595 in the case of the circular electrode of FIG. 19E and 0.601 in the case of the “symmetrical” electrode of FIG. 19F). In this situation, the “symmetrical” electrode of FIG. 19F provides an effective cross-sectional area 45% larger than the circular electrode of FIG. 19E.

In each of the above FIGS. 19B, 19D, and 19F, it is equally clear that, for a given electrode size, the shape of the effective cross-sectional area for fluid flow in the cochlea after implantation more closely mimics the shape of the effective cross-sectional area for fluid flow in the cochlea prior to implantation, leading to more natural fluid flow characteristics in the cochlea relative to the examples set forth in FIG. 19A, 19C, and 19E.

The various aspects of the present disclosure may also be applied to non-perimodiolar, or straight electrodes (in that they are pre-disposed to a straight shape and the cochlear forces them to curve and the placement is thus on the outer wall of the scala tympani).

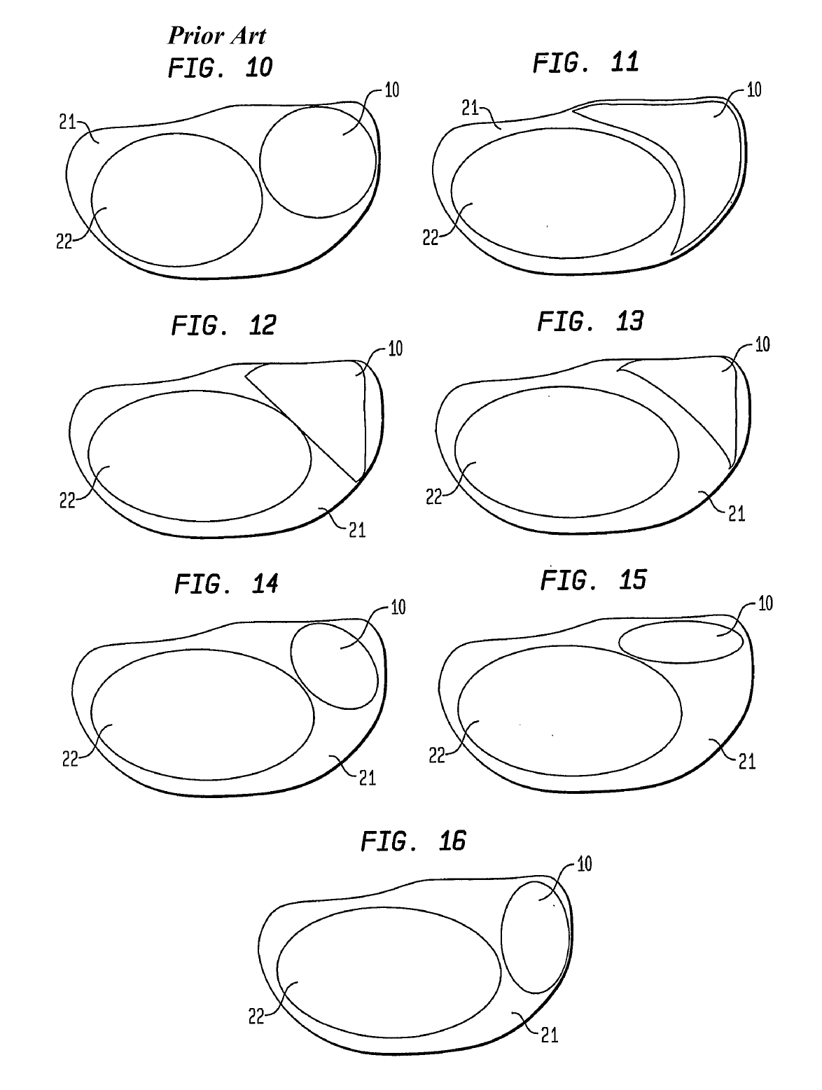

FIG. 10 shows the scala tympani 21 with a conventional circular non-perimodiolar (e.g., straight) electrode 10, and shows the effective cross-sectional area 22. In FIG. 11, the straight electrode of FIG. 10 is replaced with an improved non-perimodiolar (e.g., straight) electrode 10 shaped in accordance with an aspect of the present disclosure, to provide for a greater effective cross-sectional area 22. Note that while the circular electrode in FIG. 10 has substantially the same cross-sectional area as the improved electrode in FIG. 11, the effective cross-sectional area for the improved electrode is increased by more than 28% compared to the circular electrode. Further note that, while the circular electrode in FIG. 10 has substantially the same cross-sectional area as the improved electrode in FIG. 11, the shape of the effective cross-sectional area for fluid flow in the cochlea after implantation in FIG. 11 more closely mimics the effective cross-sectional area for fluid flow in the cochlea prior to implantation (relative to that of FIG. 10), leading to more natural fluid flow characteristics in the cochlea.

One advantage of the straight electrode design is that the cross section can be more easily tailored (i.e., made symmetrical) to facilitate large effective cochlea areas without siding the electrode (i.e., requiring a different electrode for left or right cochleas).

FIGS. 12 and 13 show various embodiments for this aspect. In FIG. 12, the straight electrode 10 provides larger effective cross-sectional area 22 for maximum fluid flow and is left and right cochlea suitable. FIG. 13 shows the straight electrode 10 with a concave inner edge again maximizing fluid flow. This arrangement is also left and right cochlea suitable.

The non-perimodiolar design is also applicable to various elliptical configurations as shown in FIGS. 14, 15, and 16.

According to a further aspect of the present disclosure, the various principles described herein may also be applied to the traverse of the electrode between cochleostomy and the start of the perimodiolar portion of the electrode in the basal turn. This design avoids blocking of the cochlea duct and thus allows for improved fluid flow in the channel.

Traditionally, cochleostomies are placed on the lateral wall. If a full flow characteristic is to be maintained then the electrode would need to cross the cochlear duct to the modiolar wall without disrupting the channel. This may be done by travelling on the scala tympani floor until the modiolar wall is reached.

The preservation of residual hearing is assisted by the present disclosure through keeping the basal turn clear for fluid flow. In conventional designs, perimodiolar electrodes travel from the cochleostomy to the inner wall and in doing so, block the fluid flow across the traverse. The portion of the electrodes travelling across this traverse are typically at their largest diameter in this region.

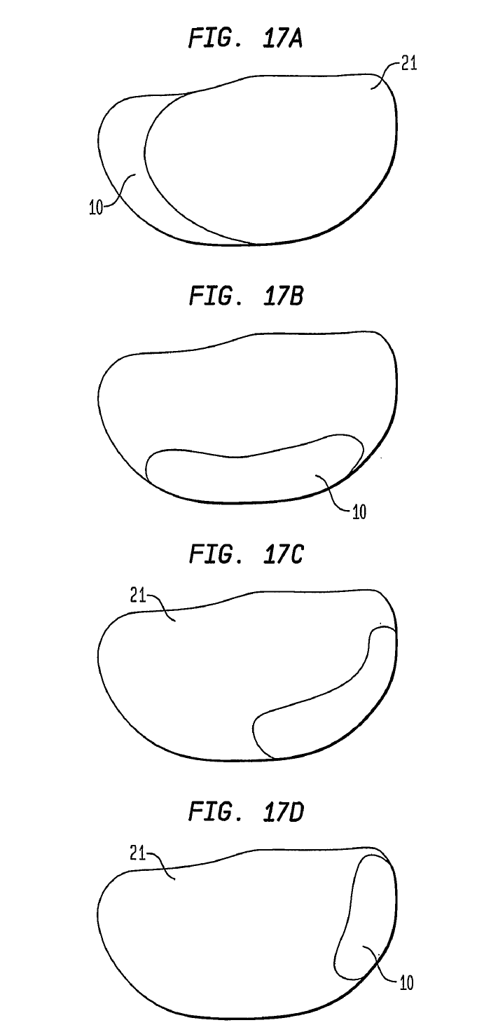

FIGS. 17A, 17B, 17C, and 17D illustrate how an improved electrode 10, designed according to the principles of the present disclosure, varies through the traverse region. Shown in FIG. 17A is the electrode 10 in its perimodiolar placement on the left hand side (LHS) of the scala tympani 21. FIG. 17D shows the electrode 10 at its entry. Through the transverse the electrode 10 follows the bottom wall of the cochlea (FIGS. 17B and 17C). In a further alternative form, the electrode 10 can follow the top of the scala tympani, rather than the bottom as described above.

A pre-curved electrode has a minimum volume required to maintain shape. There is no requirement for shape retention across the traverse (electrode typically straight). As a further alternative, the electrode can be reduced in diameter across this region and thus facilitate fluid flow.

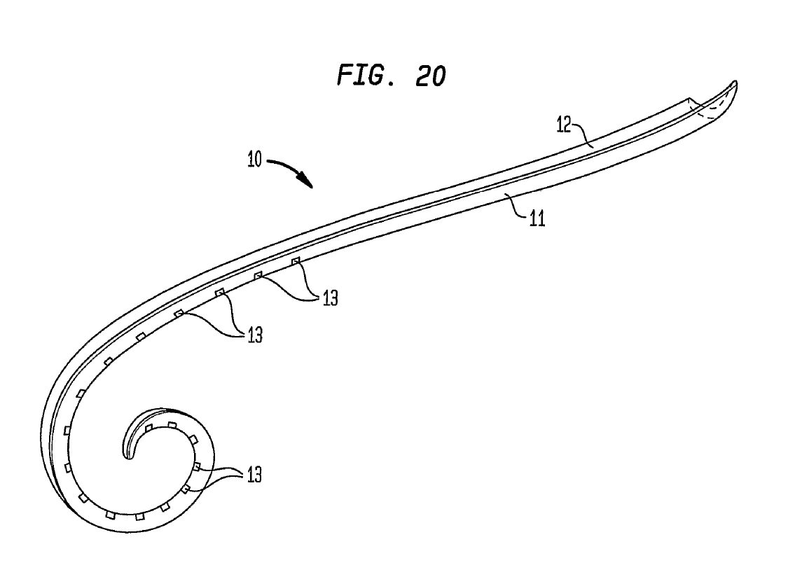

FIG. 20 shows a perspective view of an electrode 10 that has a cross section similar to that of the electrode shown in FIG. 4. Here it can be seen that electrode 10 has an electrode contact supporting surface 11 for supporting electrode contacts 13 and a “fluid side” surface 12 that will come into contact with the cochlea fluid when the implant is in situ.

It will be understood that the above has been described with reference to particular embodiments and that many variations and modifications may be made within the scope of the different aspects of the present disclosure.

For example the various aspects of the present disclosure are equally applicable to electrodes and leads for medical implants other than cochlear implants.

Throughout the specification and the claims that follow, unless the context requires otherwise, the words “comprise” and “include” and variations such as “comprising” and “including” will be understood to imply the inclusion of a stated integer or group of integers, but not the exclusion of any other integer or group of integers.

The reference to any prior art in this specification is not, and should not be taken as, an acknowledgement of any form of suggestion that such prior art forms part of the common general knowledge.

What is claimed is:

1. A cochlea electrode, at least a portion of the electrode comprising:
a first side that is curved in a lateral dimension so as to substantially conform to a side wall of a scala upon implantation of the electrode into a cochlea,
a second side that is shaped so as to preserve a natural acoustic pathway within the scala upon implantation of the electrode into the cochlea.

2. The electrode of claim 1, wherein the natural acoustic pathway is formed by a fluid side of the electrode, a side wall of the scala opposite the electrode, a top wall of the scala, and a bottom wall of the scala.

3. The electrode of claim 1, wherein the natural acoustic pathway is formed in part by a fluid side of the electrode, and wherein the fluid side is substantially concave.

4. The electrode of claim 1, wherein the natural acoustic pathway is formed in part by a fluid side of the electrode, and wherein the fluid side is substantially convex.

5. The electrode of claim 4, wherein the fluid side is substantially the second side.

6. The electrode of claim 1, wherein the electrode is further shaped such that a cross section formed by a fluid side of the electrode, a side wall of the scala, a top wall of the scala and a bottom wall of the scala is substantially circular.

7. The electrode of claim 1, wherein the electrode is substantially peri-modiolar.

8. The electrode of claim 7, wherein the electrode is pre-curved.

9. The electrode of claim 1, wherein the electrode comprises a straight electrode

10. The electrode of claim 1, wherein the sidewall comprises the lateral side wall of the scala.

11. The electrode of claim 1, wherein the sidewall comprises the modiolar side wall of the scala.

12. The electrode of claim 1, wherein the electrode is substantially crescent-shaped in cross section.

13. The electrode of claim 1, wherein the electrode is substantially oval in cross section.

14. The electrode of claim 1, wherein the electrode is substantially semi-circular in cross section.

15. A method of forming at least a portion of a cochlea electrode, the method comprising:
forming a first side of the portion of the cochlea electrode so that it is curved in a lateral dimension and substantially conforms to a side wall of a scala upon implantation in a cochlea; and
forming a second side of the portion of the cochlea electrode so that it is shaped so as to preserve a natural acoustic pathway within the scala upon implantation in the cochlea.

16. The method of claim 15, wherein the second side of the portion of the cochlea electrode is a fluid side of the electrode and is formed substantially concave in cross-section.

17. The method of claim 15, further comprising forming the second side of the portion of the cochlea electrode to have substantially a crescent-shape in cross-section.

18. The method of claim 15, wherein the natural acoustic pathway is formed by a fluid side of the portion of the cochlea electrode, a side wall of the scala opposite the electrode, a top wall of the scala, and a bottom wall of the scala.

19. The method of claim 15, wherein the portion of the cochlea electrode is a straight electrode, the method further comprising implanting the straight electrode into the cochlea and causing the first side portion of the straight electrode to conform to an outer side wall of the scala and the second side portion of the straight electrode to preserve a natural acoustic pathway within the scala.

20. The method of claim 15, wherein the portion of the cochlea electrode is a pre-curved electrode, the method further comprising implanting the pre-curved electrode into the cochlea and causing the first side portion of the pre-curved electrode to conform to an inner side wall of the scala and the second side portion of the pre-curved electrode to preserve a natural acoustic pathway within the scala when implanted.
